(19) United States
(12) Patent Application Publication
(10) Pub. No.: US 2023/0181377 A1
(43) Pub. Date: Jun. 15, 2023

(54) FIBROUS LAYER HAVING HYDROPHILIC PROPERTIES AND A FABRIC COMPRISING SUCH LAYER

(71) Applicants: PFNONWOVENS HOLDING S.R.O., Prague 1 (CZ); PFNONWOVENS CZECH S.R.O., Znojmo (CZ); PFN - GIC A.S., Znojmo (CZ)

(72) Inventors: Pavlina KASPARKOVA, Znojmo (CZ); Michael KAUSCHKE, Chiemsee (DE); Zdenek MECL, Nový Saldorf-Sedlesovice (CZ)

(21) Appl. No.: 17/924,202

(22) PCT Filed: May 7, 2021
(86) PCT No.: PCT/CZ2021/050049

§ 371 Date: Nov. 9, 2022

(30) Foreign Application Priority Data
May 9, 2020 (CZ) PV 2020-256

(51) Int. Cl. A61F 13/15; A61L 15/26; A61L 15/24; A61L 15/28

(52) U.S. Cl. CPC A61F 13/15; A61L 15/26; A61L 15/24; A61L 15/28

(57) ABSTRACT

A fibrous layer, wherein surface of the fibres has surface energy below 50 mN/m, characterised in that the calculated strike through time coefficient (cSTT) of the fibrous layer is below 20 and the fibrous layer is bonded in its entire volume at fibre to fibre contact bonding points, wherein the specific fibre surface is the surface area of the fibres in m² per 1 m² of the fibrous layer, basis weight is the weight of the layer in kg per 1 m² of the fibrous layer, the specific void volume is the volume of empty spaces between the fibres in m³ per 1 m² of the fibrous layer.

$$cSTT = \frac{{( specific fibre surface )}^{2} \times ( basis weight )}{( specific void volume ) \times {( surface energy of fibre surface )}^{3}} \times 600$$

### FIELD OF INVENTION

The invention relates to a fibrous layer having short strike through time for liquids and being suitable for use in absorbent articles, such as absorbent hygiene articles. The invention also relates to a fabric comprising such fibrous layer.

### BACKGROUND OF THE INVENTION

Fibrous layers for absorbent articles have been described in various prior art documents, wherein specific embodiments of fibrous layers intended for topsheets, backsheets, wicking layers, core wrap layers and acquisition-distribution layers have been disclosed. In some cases, the shortest possible strike through time of some of the layers is required. The strike through time of a fibrous layer may be reduced e.g. by using fibers made of hydrophilic materials or treating the fibrous layer with a hydrophilic spin finish. However, there are other factors influencing the strike through time of a fibrous layer. The objective of the present invention is to achieve good overall hydrophilic properties in a fibrous layer, such as spunmelt nonwoven fabric, the hydrophilicity being meant not only for water as such, but also at least for physiologic solutions, hence the properties are called “philic” or “liquid-philic” properties in this application. Good “philic” properties are understood to be a combination of a fast intake of a liquid into a fabric with the surface of the fabric remaining dry after the liquid has passed through the fabric.

Fast intake can be expressed, for example, by the “Strike Through Time” (STT) measurement followed by a so-called “rewet” measurement taken on the surface of the fabric. The shorter the STT, the faster is the intake of the liquid. The smaller the rewet, the drier will be the surface.

It is known, that the “liquid-philicity” of a surface can be expressed by means of the difference between surface energy of the liquid and the surface energy of the solid surface. For example, the surface energy of water is 72.8 mN/m at 20° C. A drop of water that is placed on a surface with a lower surface energy (for example polypropylene with a surface energy of around 30 mN/m) will act to touch the solid surface as little as possible, thus forming a contact (theta) angle between 90° and 150° (see FIG. 1) and thus the surface is designated as hydrophobic. When the contact (theta) angle is over 150°, the surface is designated as superhydrophobic (e.g. teflon). Hydrophilic surface supports/enables the drop to cover a greater area with a resulting contact (theta) angle that is less than 90°.

The described solid surface may be, for example, a foil (film). A nonwoven fabric consists of fibres with free space between them, or so-called “void space”, or void volume. In describing the hydrophilic and hydrophobic behaviour of a fabric, one must also consider the so-called “capillary effect”. In general, it can be said that, a hydrophobic fibre surface creates a negative capillary effect that prevents, or at least significantly limits, the liquid from entering the fabric's void space.

For example, a spunmelt nonwoven fabric is often produced from polyolefins, where the fibre surface consists of polypropylene or polyethylene, where both materials have a surface energy close to 30 mN/m). A spunbond or meltblown fabric made from such polymers is generally hydrophobic in nature. In the case where hydrophilicity is required, the fabric is treated with a so-called “spin finish” (applied, for example, by means of a kiss roll or spray) or the polymer surface energy is raised by a proper in-polymer additive or by means of a physical treatment (e.g. corona, plasma).

It is known that the ability of a fluid to penetrate a porous material depends on the size of the material's pores. The pores in a nonwoven material structure can be described as capillaries. The behaviour of fluids in capillary structures is described by the Laplace equation

$$capillary pressure = - \frac{2 * ( liquid surface tension γ ) * cos ( contact angle θ )}{capillary radius r}$$

In general, if the capillary surface is hydrophilic (higher surface energy and lower contact angle), it can be assumed that the smaller the diameter of the capillaries, the better will be the fluid transport (wicking effect), whilst the movement of the fluid is further improved when the fluid moves from larger to smaller capillaries. Conversely, it can be assumed that a lower surface tension leads to a decreased wicking speed and distance.

When the phenomena that take place when a fluid comes into contact with a (nonwoven) structure are analysed, the effect of the structure's surface characteristics and structural characteristics can be seen in combination. The hydrophilicity of a fibre's surface and the capillarity of the nonwoven structure (i.e. its density, void space, pore size, pore size distribution over the Z-direction, etc.) plays a role in affecting the final time required for fluid absorption (e.g. measured as strike through time).

The multitude of factors influencing the process of fluid penetration into the nonwoven structure makes it extremely difficult to calculate or predict the absorption properties of nonwoven materials. Thus designing a nonwoven fabric structure with excellent strike through time properties based on the prior art knowledge is cumbersome, time-consuming and very expensive, because—to provide various experimental samples of fabric—very expensive experiments have to be performed involving very high costs of use, setting and re-setting of the expensive machines for nonwoven fabrics production.

### SUMMARY OF THE INVENTION

With surprise, it has been found that respective behaviour of a nonwoven layer of fibres or a nonwoven web can be described by means of a combination of parameters such as surface energy, basis weight, void space and estimated fibre surface area to predict a calculated strike through time and thus enable the design of unexpected combinations of fabric structures and polymer compositions that achieve the desired properties when in contact with a liquid. Essentially, this surprisingly accurate method enables a fabric to be engineered to provide target properties without time- and resource-consuming experiments having to be undertaken.

Hence, the prior art drawbacks are eliminated by a fibrous layer, wherein surface of the fibres has surface energy below 50 mN/m, characterised in that the calculated strike through time coefficient (cSTT) of the fibrous layer is below 20 and the fibrous layer is bonded in its entire volume at fibre to fibre contact bonding points, wherein

$$cSTT = \frac{{( specific fibre surface )}^{2} \times ( basis weight )}{( specific void volume ) \times {( surface energy of fibre surface )}^{3}} \times 600$$

wherein the specific fibre surface is the surface area of the fibres in m² per 1 m² of the fibrous layer,
basis weight is the weight of the layer in kg per 1 m² of the fibrous layer,
the specific void volume is the volume of empty spaces between the fibres in m³ per 1 m² of the fibrous layer.

The prior art drawbacks are also eliminated by a fabric comprising the above mentioned fibrous layer, wherein the fibrous layer forms a first fibrous layer (A) and the fabric comprises a second fibrous layer (B) arranged adjacent the first fibrous layer (A), wherein the difference between the calculated strike through time coefficient cSTT of the first fibrous layer (A) and of the second fibrous layer (B) is at least 0.5, preferably at least 1.0, more preferably at least 1.5, most preferably at least 2.0.

Prior art drawbacks are also eliminated by a fibrous structure comprising at least two layers, one of them comprising cellulosic crosslinked, stiffened and curled fibres and another one comprising synthetic fibres, characterised in that
the cellulosic fibres exhibit fibrils in their cross section and the synthetic fibres comprise homogeneous polymer or polymers in its cross section, and
the cellulosic fibres have an average length of maximum 8 mm or less and
the synthetic fibres have an average length larger than 80 mm and
at least one of the layers contains bonding material.

Preferred embodiments are defined in the dependent claims.

Prior art drawbacks are also eliminated by an absorbent article, comprising topsheet, backsheet and at least one intermediate nonwoven fibrous layer arranged between the topsheet and the backsheet and comprising polymeric superabsorbent particles, wherein at least one of the topsheet, backsheet and the intermediate nonwoven fibrous layer is formed by the above specified fibrous layer, or by the above specified, or by the above specified fibrous structure.

When a liquid medium comes into contact with a fibrous surface, it can be observed in order to determine how it reacts with this surface, i.e. whether it remains on the fabric's surface (defining such a fabric as “phobic” to the liquid) or whether it enters the fibrous structure (defining such a fabric as “philic” to the liquid). The speed of the liquid's entry determines the degree of “philicity”, i.e. from slightly philic to very philic). Various fabric applications require various levels of “philicity”. For example, when a fabric is required to be able to quickly remove a liquid from its surface a higher level of philicity is required. An important factor is also the intended behaviour of the liquid inside the fabric. For example, various wipes are designed to retain the liquid inside them (to remain wet), conversely, for example, in certain hygiene applications (e.g. a topsheet in disposable hygiene products) the fabric should acquire the liquid and transport it to another part of product (quickly become dry again) or (e.g. at the Acquisition Distribution Layer (ADL) in hygiene disposable products) the fabric should draw the liquid inside, slow down its flow, then distribute it into a broader area and transfer it to another part of product (and become dry again). Each of the above mentioned examples requires a different character of liquid-philicity. And person skilled in the art is able to name many other examples with specific requirements for fibrous layer liquid-philicity and with respect to water specific requirements for fibrous layer hydrophilicity.

For example, for some applications it can be advantageous when the fibre itself is able to absorb liquid into itself. For example, cotton or pulp fibres can swell by absorbing water (see FIGS. 2a to 2c, wherein FIG. 2a is a native cotton fibre, FIG. 2b is the fibre swollen by 1-butanol and FIG. 2c is the fibre swollen by water).

In fabric applications, where the dryness is important, swollen fibres or fibres absorbing liquid into themselves might be unwelcome. Conversely, fibres with “closed” surfaces do not absorb liquid. Water is a special case, where so-called “hygroscopic” polymers draw a certain amount of water from the environment (e.g. from ambient humidity) and build the water molecules into their polymeric structure (e.g. PET, nylon, PLA etc.). Such hygroscopic polymers that absorb only a small amount of water vapour and that do not swell significantly in water or water-based solutions are considered to be polymers with closed surfaces.

The fibrous layer according to the invention comprises fibres that do not swell by drawing liquid into themselves. In a preferred embodiment, the fibrous layer according to the invention comprises fibres that do not swell in water, water-based solutions or body fluids.

The “philicity” of a flat surface is given by the difference between the surface energy of the flat surface and the surface energy of the liquid. However, generally the fibrous layer consists of fibres and free space between the fibres, or so-called void space. In other words, one can speak of fibrous layer porosity (as expressed in the Laplace equation). The bulkier the fabric is and the bigger the pores are and thus the smaller is the effect of the fibre polymer surface energy. When the pores are large enough, the liquid can simply pass through the fibrous layer without any actual interaction with the fibre surface. Conversely, with a decreasing pore diameter, the liquid is forced to interact with the fibre surface and thus the fibre surface energy influences the interaction between the fibrous layer and liquid more. At certain pore size, when the surface energy difference between the polymer and the liquid is sufficiently large, the so-called capillary effect can be observed, and it can be either positive (draws the liquid into the pores) or negative (pushes the liquid out of the pores). When the surface energy difference is more or less comparable, then no or only a very small capillary effect is observed (see FIGS. 3a to 3c).

With pure water having a nominal surface energy of 72.8 mN/m, the fibrous layer according to the invention generates a neutral or very low negative capillary effect that can be overcome by a small force, for example, the force during the Strike Through Time (STT) measurement is sufficient to overcome the low negative capillary effect. Preferably, the fibrous layer according to the invention provides a strike through time value lower than 20 seconds, preferably lower than 15 seconds, preferably lower than 10 seconds, with advantage lower than 5 seconds.

The Laplace equation as used in the textile industry disregards the length of the pores and their tortuosity, which are parameters that are extremely important for describing fabric behaviour of fibres that have a lower surface energy than that of the applied liquid. Also, the equation does not consider the specific case of bulky fibrous layers, especially bulky fibrous thermobonded layers, where the pores typically provide a rather large irregular radius based on the given space between the fibres.

Not to be bound by theory, it is believed that in this invention a specific relationship between the fibre surface, its surface energy and the void space of the fabric for defined basis weights has been found that allows the hydrophilic properties of the nonwoven fabric to be set to the desired level as expressed by the “calculated STT coefficient”, i.e. “cSTT” coefficient for water and water solutions:

$$cSTT = \frac{{( specific fibre surface )}^{2} \times ( basis weight )}{( specific void volume ) \times {( surface energy of fibre surface )}^{3}} \times 600$$

wherein the specific fibre surface is the surface area of the fibres in m² per 1 m² of the fibrous layer,
basis weight is the weight of the layer in kg per 1 m² of the fibrous layer,
the specific void volume is the volume of empty spaces in m³ per 1 m² of the fibrous layer.

Where:

The surface energy of the fibre surface (measured using a fibre surface polymer composition—a small plate that is prepared from the polymer composition and its surface energy is measured by the drop method with 3 liquids and the Owens-Wendt-Rabel-Kaelble calculation (OWRK model), or in case of a fibre structure, the Washburn method can be used). As used herein the surface energy is meant to be the surface energy measured at 20° C. and using OWRK model for polymers and using the Washburn method for cellulose.

Typical surface energy values are readily available, however, for the purpose of the cSTT calculation it is advantageous to have exact values. Some examples of various thermopolymer grades can be seen in table 1:

TABLE 1

| | | Nominal surface energy |
|---|---|---|
| Polymer grade/fibre type | Producer | (mN/m) |
| Crosslinked, curled and | International | 46.2 ± 0.5 |
| stiffened fibres | Paper (former | |
| | Weyerhaeuser) | |
| PET copolymer type | Trevira | 45.8 ± 2.5 |
| RT5023 | | |
| PET type 5520 | Invista | 40.8 ± 6.7 |
| PET copolymer type | Invista | 36.7 ± 1.4 |
| 701k | | |
| PLA type 6100D | Nature Works | 42.9 ± 5.1 |
| PLA type 6202D | Nature Works | 42.8 ± 1.7 |
| PP Tatren HT 2511 | Slovnaft | 29.6 ± 4.0 |
| PE Aspun 6834 | Dow Chemicals | 32.7 ± 1.7 |
| Softblend | — | 29.4 ± 1.6 |
| (PP + copo PP/PE + | | |
| erucamide) | | |

There are well known and commonly used bi-component or multi-component fibres in the industry. In the case where the surface is formed using a single polymer (for example, the so-called core-sheath cross section arrangement), the surface energy of the surface polymer is used in the equation. In the case where the surface is formed by from more than one polymer, the surface ratio of all the polymers shall be set and the surface energy of the fibre is calculated as a weighted average.

The specific fibre surface can be estimated theoretically from the median fibre thickness and the total mass volume of the fabric—the greater the surface area, the greater the surface effect (or capillary effect in the fabric). In general, finer fibres have a greater surface area than coarser fibres when considering webs having the same basis weight. The specific fibre surface is calculated as the surface area of the side of a cylinder formed from all the mass in 1 square meter of fabric, the diameter of the cylinder is equal to the median fibre thickness.

$$fibre surface = ( fibre circumeference ) \times ( fibre length )$$

$$specific fibre surface [ \frac{{m}^{2}}{{m}^{2}} ] = \frac{4 \times ( layer basis weight [ \frac{kg}{{m}^{2}} ] )}{( fibre density ρ [ \frac{kg}{{m}^{3}} ] ) \times ( fibre diameter [ m ] )}$$

The fibre length is calculated based on the known basis weight and on the surface area of the fibre cross section. The formula is intended for fibres with round cross section. In the case of a different cross section shape, a person skilled in the art can find an appropriate equation for calculating the surface area of the cross section of the fibre, fibre circumference and fibre length according to current situation.

The fibre composition density in the equation can be measured according to the norm ISO 1183-3:1999 or estimated from its composition as a proportional average of the density of each component.

Especially for fabrics with a very broad or unequal distribution of fibres, the estimation based on average fibre diameter can be far from reality. For such cases, it is suggested to divide the fibre thickness measurements into logical groups by size, then calculate the theoretical fibre surface area separately for each group and then add them together. Same method can be applied for different layers formed, for example, from slightly coarser and finer fibres. For example, when a fabric with a 15 gsm basis weight is formed from two layers of spunbond fibres with no definite border between the layers, the fibre thickness measurement in the fibre cross section is provided showing two narrow peaks in fibre thickness. One at 25 microns and the second at 35 microns, the second value constitutes two thirds of the total basis weight of these two layers. The theoretical fibre surface then should be calculated separately for the first peak (median approx. 25 microns, ⅓ of fabric mass=5 gsm) and for the second peak (median approx. 35 microns, ⅔ of fabric mass=10 gsm) and then added together (15 gsm).

The specific void space herein refers to the total amount of void space i.e. void volume in a material in 1 m2 of fabric.

$$specific void space [ \frac{{m}^{3}}{{m}^{2}} ] = spefific bulk volume [ \frac{{m}^{3}}{{m}^{2}} ] - specific mass volume [ \frac{{m}^{3}}{{m}^{2}} ] == \frac{( fabric thickness [ m ] ) * 1 [ m ] * 1 [ m ]}{{1 [ m ]}^{2}} - \frac{basis weight [ \frac{kg}{{m}^{2}} ]}{fiber composition density ρ [ \frac{kg}{{m}^{3}} ]}$$

In the equation, the basis weight is expressed in kg/m2. The basis weight of a layer of fibres in a composite can be taken from the production process settings, or be an estimation, where the composite is delaminated and the basis weight of each layer can be distinguished separately.

Coefficient “k” (in the cSTT calculation) is added to the equation to move the number range closer to the actual STT values. K=600.

Not to be bound by a theory, it is believed that the size of the fibre surface area in combination with the void space and basis weight expresses the structure of a nonwoven fabric, especially the structure of a bulky nonwoven fabric, better than the capillary radius. Void space together with the basis weight describes the amount of mass in the defined space, and the size of the fibre surface area defines the distribution of the mass. The same mass in the same space with a small fibre surface area size provides coarse fibres with large pores between them, conversely a large fibre surface area size provides fine fibres with small pores. The size of the fibre surface area provides the surface with which the liquid is forced to interact, and the void space provides the space where the liquid can flow through the fabric.

For a material composition, this void space can also be expressed as the bulkiness of the fabric. The higher the void space, the bulkier the fabric is. Bulkiness, rather bulk density is expressed as a unit of weight per unit of volume, and thus is dependent on fibre material density, and conversely, void space represents the void volume between fibres in the fabric and hence is independent of fibre density.

All the aforementioned values can be measured or determined for a single layer or independently for any individual layer contained within a composite consisting of multiple layers.

The cSTT coefficient should be understood as an estimation of the strike through time in seconds. The lower the value, the faster is the real strike-through-time of the fabric. The cSTT coefficient predicts the real strike through time of a liquid as defined in the STT method (water with 0.9% NaCl). It can be understood as an estimation for liquids with a surface energy of 80-60 mN/m, preferably for water solutions with a surface energy of 80-60 mN/m, with advantage for water solutions with a surface energy of 76-66 mN/m. The behaviour of liquids with a higher or lower surface energy in the fibrous layer can be also predicted by the cSTT coefficient, however, it should be interpreted with respect to the following knowledge. Lower surface energy of a liquid speeds up the STT, so that real STT value would be lower than cSTT. Higher liquid surface energy of a liquid slows down the STT, so that real STT value would be higher than cSTT.

A hydrophilic fibrous layer according to the invention has a calculated cSTT coefficient that is lower than 20, preferably lower than 15, preferably lower than 10, with advantage lower than 5.

A fibrous layer according to the invention has a dry surface after the intake of a liquid. For example, the so-called Strike Through Time measurement according to the standardised testing methodology WSP 70.3 as issued by the European Disposables and Nonwovens Association (EDANA) tests the speed of liquid intake until there is no liquid on the surface=until the electric circuit provided by electrodes attached to said surface is interrupted. When the liquid disappears from the surface of the fabric, the electric circuit is interrupted.

A fibrous layer according to the invention provides a real STT coefficient that is lower than 20, preferably lower than 15, preferably lower than 10, with advantage lower than 5.

The cSTT can be used to predict the liquid-fabric behaviour for many types of polymers and fabric structures.

For example, the cSTT coefficient can be used to predict the behaviour of fabrics with a basis weight from 10 gsm, preferably from 15 gsm, with advantage from 20 gsm. The basis weight of one considered fabric or layer should not exceed 200 gsm, preferably 150 gsm, more preferably 120 gsm, even more preferably 100 gsm, more preferably 80 gsm, with advantage 60 gsm.

For example, the cSTT coefficient can be used to predict the behaviour of fabrics with a bulk density lower than 30 kg/m3, preferably lower than 25 kg/m3, with advantage lower than 30 kg/m3.

For example, the cSTT coefficient can be used to predict the behaviour of fabrics with an average fibre thickness of at least 10 microns, preferably of at least 15 microns, with advantage of at least 17 microns. The fibre thickness should not exceed 200 microns, preferably 100 microns, more preferably 50 microns.

For example, the cSTT coefficient can be used to predict the behaviour of fabrics from fibres with various cross section shapes, preferably it can be used for fibres with a round or approximately round cross section shape.

For example, the cSTT coefficient can be used to predict the behaviour of fabrics with various fibre shapes, it can be used for any non-crimped or crimped fibres, where crimped fibres are understood to be all known types of crimping, for example crimped by external force (typically staple fibres), self-crimped, controlled shrinkage driven crimping, activated crimping, etc.

For example, the cSTT coefficient can be used to predict the behaviour of fabrics bonded in their entire volume at fibre-fibre contact bonding points, as for example thermobonded by hot medium flow (e.g. air-through-bonded) or, for example, bonded by means of an adhesive added to the fibrous structure (e.g. addition of dust glue into the fabric that is activated by energy).

For example, the cSTT coefficient can be used to predict the behaviour of fabrics or layers produced from any type of short, staple or endless fibres, for example, from endless spunbond fibres, with advantage from endless air-through-bonded spun fibres. It should be noted, that the cSTT can only be used to predict the strike through time for a singular specific fabric. The cSTT values calculated for layers in a composite should not be added together. Not to be bound by the theory, it is believed that adjacent layers with possible interference or interconnection of fibres between layers with liquid present in the void space of one layer provide better and faster liquid transport into an adjacent layer than free liquid is able to enter the same fibrous layer.

The layer, according the invention, can be combined with any other layer in the final product (e.g. disposable hygiene product) or in composite fabric bonded together at fibre-fibre contact bonding points. Another layer of a material compound can be made, for example, of spun filaments of another thickness or cross section or polymer composition or surface characteristic or structure characteristics.

The air-through bonded filaments can be bi-component filaments, which have been brought to a three-dimensional shape by crimping if the cross section of the bi-component filaments is asymmetric, e.g. eC/S or in S/S configuration (so-called crimp supporting cross section or configuration). Such webs provide bulkiness or loftiness. Another way to produce bulky/lofty webs from bi-component filaments having a crimp non-supporting cross section is described in patent application WO2020103964 filed by the companies PFNonwovens Czech s.r.o., PFN-GIC a.s. and REIFENHAUSER GMBH & CO. KG MASCHINENFABRIK, where a shrink force results in the bowing/arching of filaments.

It can be seen that certain combinations of fibre surface characteristics, fibre size and free space between the fibres, can bring unexpected advantages.

In one embodiment of the invention, the fabric can be a thermobonded spunmelt nonwoven fabric as described in the patent application WO2020103964, where the bulkiness of the fabric is enhanced by controlled shrinkage of the fibres. The fabric is produced from bi-component fibres with crimp non-supporting cross sections, where at least one component on the surface works as a bonding component. For example, fibres with a surface from polypropylene, polyethylene, polylactic acid or polyethylene terephthalate can form such a fabric. Each of the aforementioned polymers has a surface energy much lower than that of water (72 mN/m) and so it is expected that the fabric will behave hydrophobically. Typically, a hydrophilic spin finish or additive is used to increase the surface energy of the fibres, or physical treatment like plasma or corona is used, to make the fabric hydrophilic and to increase the surface energy so that it is closer to that of water (for example using Silastol PHP 90 from Schill and Seilacher increases the PE surface energy from 32.7 mN/m to 52.7 mN/m and, respectively, using Silastol PHP 10 from Schill and Seilacher, increases the coPET surface energy from 45.8 mN/m to 55.2 mN/m). Surprisingly, by means of a cSTT calculation it was predicted that the bulky structure of a fabric at specific circumstances allows water or water-based solutions to behave hydrophilically even when the fibre surface energy is naturally hydrophobic and this prediction was confirmed (see examples).

For example, a nonwoven layer of fabric formed from bi-component fibres with a core comprising the first shrinkable polymer and a sheath comprising the second bonding polymer can be produced by means of a method comprising the following steps:

a) melting and feeding at least a first polymeric material and a second polymeric material having its melting point lower than the first polymeric material to nozzles of a spinning beam, wherein the nozzles are configured to produce endless filaments having all components arranged across the cross section of the filaments in a crimp non-supporting configuration, wherein the second polymeric material extends in the longitudinal direction of the filament and forms at least a part of the surface of the filament, where the filament speed is within the range of 3000 and 5500 m/min,
b) cooling of the formed filaments by means of a fluid medium having a temperature within the range of 10 to 90° C. and drawing the filaments with a draw down ratio within the range of 200-1300 to achieve a semi-stable crystalline state of at least the first polymeric material,
c) depositing the filaments onto a formation belt to form a nonwoven filamentary batt,
d) heating the nonwoven filamentary batt to a temperature within the range of 80 to 200° C. to activate shrinkage of the nonwoven filamentary batt, such that at least the polymeric material is transformed to a more stable crystalline state.

Preferably, the method further includes the step of pre-consolidating the nonwoven filamentary batt after step c) and before step d), wherein the pre-consolidation is performed by heating the filaments to a temperature within the range of 80 to 180° C., preferably 90° C. to 150° C., most preferably 110° C. to 140° C. to partially soften the polymeric material in order to create bonds of polymeric material between the mutually crossing filaments.

Preferably, in step b) the filaments are cooled and drawn through a first zone with a fluid medium having a temperature within the range of 10 to 90° C., preferably 15 to 80° C., most preferably 15 to 70° C., and then through a second zone with a fluid medium having a temperature within the range of 10 to 80° C., preferably 15 to 70° C., most preferably 15 to 45° C.

According to a preferred embodiment, the heating of the nonwoven filamentary batt in step d) is performed by exposing the batt to air having the temperature within the range of 80 to 200° C., preferably within the range of 100 to 160° C., for a period of 20 to 5000 ms, preferably 30 to 3000 ms and most preferably 50 to 1000 ms. The air is preferably driven through and/or along the batt having an initial speed within the range 0.1 and 2.5 m/s, preferably within the range of 0.3 and 1.5 m/s.

The nonwoven filamentary batt is preferably heated in step d) such that it shrinks in the machine direction and cross direction by 20% or less, preferably by 15% or less, more preferably 13% or less, more preferably 11% or less, most preferably 9% or less, and increases its thickness by at least 20%, preferably by at least 40%, more preferably at least 60%, most preferably by at least 100%.

The nonwoven filamentary batt may be heated in step d) such that the polymeric material softens to create bonds of polymeric material between the mutually crossing filaments. Or, the nonwoven filamentary batt is heated after step d) such that the polymeric material softens to create bonds of polymeric material between the mutually crossing filaments. The heating after step d) that is intended to provide bonds of polymeric material (B) may be performed using an omega drum bonding device or a flat belt bonding device or a multiple drum bonder, and/or by driving air through and/or along the nonwoven filamentary batt for a time period of 200 to 20000 ms, preferably 200 to 15000 ms and most preferably 200 to 10000 ms, wherein the air has a temperature within the range of 100° C. to 250° C., preferably 120° C. to 220° C. and an initial velocity within the range of 0.2 to 4.0 m/s, preferably 0.4 to 1.8 m/s.

Preferably, the first polymeric material and/or the second polymeric material consists of, or comprises, as the majority component, a polymeric material selected from the group consisting of polyesters, polyolefins, polylactic acid, polyester copolymers, polylactide copolymers and blends thereof; and the first polymeric material is different from the second polymeric material. According to a preferred embodiment of the invention, the first polymer melt temperature is at least 5° C. greater, preferably at least 10° C. greater than the melt temperature of the second polymer.

In one embodiment according to the invention, the first polymeric material is polyester, preferably polylactic acid or polyethylene terephthalate and the second polymeric material is polyester or co-polyester, preferably the copolymer of polylactic acid or copolymer of polyethylene terephthalate.

When performing the method according to the invention, it is advantageous, when the draw down ratio is within the range of 300-800.

In another embodiment of the invention, the fabric can be a thermobonded spunmelt nonwoven fabric formed by fibres with a crimp supporting cross section, for example S/S or eC/S, in such manner that the fibres provide a certain level of crimping and the fabric is thus bulkier than fabric from the same material composition in a crimp non-supporting cross section or without activation.

For example, a nonwoven layer of fabric formed from bi-component filaments with a first polymer and a second bonding polymer with a lower melting point, where the filaments exhibit at least 3 crimps/cm, can be produced by a method comprising the steps:

a) melting and feeding at least a first polymeric material and a second polymeric material having its melting point lower than the first polymeric material to nozzles of a spinning beam, wherein the nozzles are configured to produce endless filaments having all components arranged across the cross section of the filaments in a crimp supporting configuration, wherein the second polymeric material extends in the longitudinal direction of the filament and forms at least a part of the surface of the filament,
b) cooling of the formed filaments by means of a fluid medium having a temperature within the range of 10 to 90° C. and drawing the filaments to achieve endless filaments,
c) depositing the filaments onto a formation belt to form a nonwoven filamentary batt,
d) heating the nonwoven filamentary batt to a temperature 80-300° C., wherein the second polymer melts and creates the fibre-to-fibre bonding points.

Preferably step d) is divided into multiple areas with different heat conditions. For example, there is a separate pre-consolidation device just after the production beam together with a separate thermobonding unit. For example, the thermobonding unit can be divided into several sections with different settings.

Preferably self-crimping occurs during step b) cooling and drawing and/or during step d) heating.

Preferably, the first polymeric material and/or the second polymeric material consists of or comprises as the majority component polymeric material selected from the group consisting of polyesters, polyolefins, polylactic acid, polyester copolymers, polylactide copolymers and blends thereof; and the first polymeric material is different from the second polymeric material. According to a preferred embodiment of the invention, the first polymer melt temperature is at least 5° C. greater, preferably is at least 10° C. greater than the second polymer melt temperature.

In one embodiment according to the invention, the fibres with a crimp supporting cross section comprise the first polymeric material that is polyester, preferably polylactic acid or polyethylene terephthalate and the second polymeric material is a polyolefin, polyester or co-polyester, preferably the copolymer of polylactic acid or copolymer of polyethylene terephthalate or polypropylene or polyethylene.

In another embodiment according to the invention, the fibres with a crimp supporting cross section comprise the first polymeric material that is a polyolefin, preferably polypropylene and of a second polymeric material with a lower melting point polyolefin, preferably polyethylene or PP/PE copolymer.

In another embodiment, the component of the filaments with a crimp supporting cross section can contain additives for modifying crimping. For example, so-called nucleating agents are known to improve the crimping level of a filament and thus the bulkiness and possibly also the recovery of the fabric. Nucleating agents might be, for example, aromatic carboxylic acid salts, phosphate ester salts, sodium benzoate, talc and certain pigment colorants, e.g. TiO2.

The fibrous fabric or layer according to the invention may comprise filaments with a crimp supporting or crimp non-supporting cross section.

The mass ratio of the first polymeric material to the second polymeric material is for fibers having crimp supporting cross-section preferably 70:30 to 90:10, more preferably 60:40 to 30:70, and for fibers having crimp non-supporting cross section 50:50 to 90:10. The nonwoven fabric has preferably a basis weight of at least 5 gsm, preferably of at least 10 gsm, more preferably of at least 20 gsm, more preferably of at least 30 gsm, with advantage of at least 40 gsm and preferably not greater than 200 gsm, preferably not greater than 150 gsm, preferably not greater than 100 gsm, most preferably not greater than 80 gsm.

It is also advantageous, when the filaments have a median fibre diameter of at least 5 microns; preferably at least 10 microns; preferably at least 15 microns; most preferably at least 20 microns, and at most 50 microns; preferably at most 40 microns; most preferably at most 35 microns.

Preferably, the layer has a void volume of at least 65%; preferably of at least 75%; more preferably of at least 80%; more preferably of at least 84%; more preferably of at least 86%; more preferably of at least 88%; most preferably of at least 90%.

Preferably the layer has a recovery of at least 0.8 (which corresponds to 80% recovery of the original thickness), preferably of at least 0.82, more preferably of at least 0.84, most preferably of at least 0.85.

For example, a bulky air-through-bonded nonwoven fabric is formed predominantly using bi-component fibres with a C/S cross section,
where the core is formed predominantly from polyester or polyamide and,
the sheath predominantly from a polymer with a melting point at least 10° C., preferably at least 5° C. lower than the core polymer and,
surface energy in the range of 40-50 mN/m, preferably in the range of 42-48 mN/m, with advantage of 44-46 mN/m, for example polyester (e.g. PET, PLA), polyamide, copolyester (e.g. coPET, coPLA), copolyamide and,
have a core/sheath mass ratio of 30:70 to 90:10, preferably 40:60 to 70:30 and
have a void volume of at least 65%; preferable of at least 75%; more preferably of at least 80%; more preferably of at least 84%; more preferably of at least 86%; more preferably of at least 88%; with advantage of at least 90% and
a basis weight of 5 to 200 gsm, preferably 10 to 100 gsm, preferably 20 to 80 gsm and,
have a strike through time (STT) lower than 20 sec, preferably lower than 15 sec, more preferably lower than 10 sec, even more preferably of lower than 5 sec.

For example, a bulky air-through-bonded nonwoven fabric formed predominantly from the bi-component fibres with eC/S or S/S cross section,
where one component is formed predominantly from the first polymer, preferably polyester or polyamide or polyolefin and,
the second component is formed predominantly from a second polymer with a melting point at least 10° C., preferably at least 5° C. lower than that of the first component polymer and,
proportional average surface energy of the fibre as calculated from the polymer surface area ratio is in the range of 40-50 mN/m, preferably in the range of 42-48 mN/m, with advantage of 44-46 mN/m, for example, the fibre surface comprises polyester (e.g. PET, PLA), polyamide, copolyester (e.g. coPET, coPLA), copolyamide, polyolefin (PP, PE and their copolymers) and,
have a component/component mass ratio of 30:70 to 90:10, preferably 40:60 to 70:30 and,
have a void volume of at least 65%; preferable of at least 75%; more preferably of at least 80%; more preferably of at least 84%; more preferably of at least 86%; more preferably of at least 88%; with advantage of at least 90% and,
a basis weight of 5 to 200 gsm, preferably 10 to 100 gsm, preferably 20 to 80 gsm and,
have a strike through time (STT) lower than 20 sec, preferably lower than 15 sec, more preferably lower than 10 sec, even more preferably lower than 5 sec.

In one embodiment of the invention, one layer or fabric can be made from cellulose fibres known in the hygiene industry as “fluff pulp” but treated in a way that results in the crosslinking on their surfaces. Additional treatment steps result in the curling and stiffening of these crosslinked cellulose fibres. For example, the cellulose fibres can be treated by a citric acid technology, but a person skilled in the art will appreciate also other technologies that are suitable for cellulose crosslinking. The citric acid technology can bring the advantage of a specific pH value. A lower pH value of such a layer can be advantageous, for example, in disposable hygiene product applications, where the acidic environment of the layer can buffer the alkaline ammonia from decomposing urine and thereby protect the user's skin. Disposable hygiene products containing such a layer might extend the time of use of the product.

Crosslinked cellulose is typically known for its hydrophilic but “closed for water” fibre surface and its behaviour when in contact with aqueous fluids, where it may be considered similar to thermoplastic polymers, e.g. polyolefins or polyesters. For example, in FIGS. 4a and 4b one can see that dry fibres (4a) are the same as fibres in the water (4b).

The surface energy of crosslinked cellulose might vary significantly, where some grades might be, for example, comparable to higher surface energy PLA or PET grades. For example, the crosslinked, curled and stiffened fibres offered by International Paper (former Weyerhaeuser) have a surface energy of 46.4 (+−0.5) mN/m, which is fully comparable, for example, with the PET copolymer type RT5023 from Trevira.

For example, the 40 gsm airlaid layer formed using crosslinked cellulose fibres with an average thickness of 25.33 microns (fibre surface area of 4.16 m2/m2) with a surface energy of 46.4 mN/m and a thickness of 2.2 mm (0.0022 m3/m2 of void space) provides the cSTT of 1.86 and also in reality the layer sucks in the liquid very quickly with a dry surface after liquid absorption.

For some applications, the embodiment of the invention is preferably such, that the surface energy of the fibres forming the nonwoven fabric comprising fibre-to-fibre bonding points is in the range of 30-35 mN/m, as for example the surfaces formed by polyolefins, e.g. polypropylene, polyethylene, their copolymers or blends. More specifically, such embodiments may be preferably as follows:
For some applications, it is preferable, that the basis weight of such fabric is in the range of 10-40 gsm and the specific void space, for example, can be in the range of 2.0-2.5 dm³/m² (0.0020-0.0025 m³/m²) and the specific fibre surface is below 8.4 m²/m², preferably below 7.3 m²/m², more preferably below 6.0 m²/m², with advantage below 4.2 m²/m², but higher than 0.6 m²/m².
For some applications, it is preferably, when the basis weight of such fabric is in the range of 10-40 gsm, the specific void space is in the range of 2.5-3.0 dm³/m² (0.0025-0.0030 m³/m²) and the specific fibre surface is below 10.6 m²/m², preferably 9.1 m²/m², more preferably below 7.5 m²/m², with advantage below 5.3 m²/m², but higher than 0.6 m²/m².
For some applications, it is preferable, that the basis weight of such fabric is in the range of 10-40 gsm, the specific void space is in the range of 3.0-3.5 dm³/m² (0.0030-0.0035 m³/m²) and the specific fibre surface is below 12.7 m²/m², preferably below 11.0 m²/m², more preferably below 8.9 m²/m², with advantage below 6.3 m²/m², but higher than 0.6 m²/m².

According to a preferred embodiment of the invention, the layer or web (A) according to the invention can be combined with another layer or web (B). For example, layer B can be formed from endless fibres (e.g. spunmelt or spunbond technology), and, for example, layer B can be formed from staple fibres (e.g. carded technology), for example, layer B can be formed from short fibres (e.g. airlaid technology). Layer B may fulfil all the conditions defined for layer A but, nevertheless, provide a slower strike through time, which can be predicted by cSTT. The layers A and B can differ in structure, polymer composition, fibre shape, fibre size, type of fibre cross section, etc. The composite can comprise one or more layers A according to the invention and one or more layers B that may or may not be according to this invention. The webs or layers A and B can be bonded together, for example, on their adjacent surfaces using added adhesive or by the use of a bonding polymer contained in any of the layers A and/or B. The fibres of layer A and B may interfere with each other close to the adjacent layer surfaces. For any of layers A, B, the cSTT coefficient can be calculated and certain hydrophilic properties of the composition can be advantageous for certain applications.

For example, it can be advantageous to combine two layers A, B, adjacent to each other, where the cSTT for layer A is different than the cSTT for layer B, preferably where the difference in cSTT for A and B layers is at least 0.5, preferably at least 1.0, more preferably at least 1.5, with advantage at least 2.0.

Not to be bound by the theory, it is believed that the combination of a layer comprising principally endless fibres (for example spunbond type with a minimum length of 80 mm) and a layer comprising short fibres (for example cellulose fibres with an average length of maximum 8 mm) can bring even more advantages.

For example, the fibre structure, homogeneity or regularity of a layer of endless fibres is better than a layer consisting of short fibres. Short fibres have “clusters” of fibres (for example cellulose fibres) leading to the material density being twice the average specific weight in certain spots and then sparser in other spots with a density of less than 0.5 of the average specific weight. This can improve the acquisition of fluids in the layer. Conversely, the fibrous layer of endless fibres with better homogeneity provides better conditions for fluid distribution.

For example, short fibres have endings or narrow loops that can point or be inserted into a void space in the bulky structure of the fibrous layers of endless fibres, which then helps the liquid to enter it, so that the acquisition of the layer of endless filaments is improved. For example, layer A from cross-linked cellulose fibres can be combined with layer B from bulky spunbond air-through bonded nonwoven fibres with PET, coPET, PLA, coPLA, PP, PE or their copolymers present on fibre surface. For example, a 40 gsm fabric from crosslinked cellulose with a cSTT below 2 is able to absorb liquid extremely fast and provide it to layer B, which has a lower cSTT.

The border area between webs or layers A and B slows down the passage of the liquid through the fabric. As, for example, in hygiene absorbent products, the liquid typically enters the product (e.g. diaper) in a relatively small area and needs to be rapidly absorbed into the product, where it can subsequently be distributed to the absorbent, thus it can be advantageous to slow down the passage of the liquid through the fabric composite structure and allow it to distribute more across the fabric plane (e.g. in the so-called CD and MD directions). This allows to the rewetting surface to be kept relatively small and to distribute the fluid to a much larger volume of absorbent material, which finally results in a lower rewet. In this way, both the wet surface in contact with wearer's skin and the amount of liquid returning to the skin are reduced.

For example, it can be advantageous to combine two layers A, B, adjacent to each other, where the cSTT for layer A is different to the cSTT for layer B, preferably where the difference in cSTT for layers A and B is at least 2.0, preferably at least 4.0, more preferably at least 6.0, with advantage at least 10.0. For example, layer A with a fibre surface from PLA or coPLA can be combined with layer B of bulky spunbond air-through bonded nonwoven fibres with PET, coPET, PP, PE present on the fibre surface. The PLA or coPLA surface is able to absorb liquid extremely fast, pass it to the AB border and since layer B absorbs the liquid more slowly, it has time to redistribute it across the border plane.

In one embodiment of the invention, it can be advantageous to combine two layers A, B, adjacent to each other, where the fibre surface tension in layer A is not higher than 50 mN/m and the cSTT for layer A is lower than the cSTT for B layer, preferably where the difference in cSTT for layers A and B is at least 10.0, preferably at least 15.0, more preferably at least 20.0, with advantage at least 25.0.

In one embodiment of the invention, layer A comprises fibres with a surface tension higher than 50 mN/m. For example, the fabric can be made hydrophilic by means of a spin finish. For example, table 2 shows the surface energy change after hydrophilisation using the spin finish PHP 90 produced by Schill and Seilacher.

TABLE 2

| | | | Surface energy |
|---|---|---|---|
| | | | after spin finish |
| | | Surface energy | treatment |
| Polymer grade | Producer | (mN/m) | (mN/m) |
| PET type 5520 | Invista | 40.8 ± 6.7 | 55.4 |
| PLA type | Nature Works | 42.8 ± 1.7 | 55.2 |
| 6202D | | | |
| PE Aspun 6834 | Dow Chemicals | 32.7 ± 1.7 | 52.7 |

The spin finish treatment is normally performed by applying the spin finish solution to the fabric and then drying the fabric by hot air. When further liquid is applied to the fabric, as for example in an absorbent hygiene product during use, a certain amount of the spin finish substances may be diluted by the liquid and decrease its surface energy, which leads to a higher absorption to the subsequent layer B of the fabric.

In one embodiment of the inventions, it can be advantageous to combine two layers A, B, adjacent to each other, where layer A was treated with a spin finish and the cSTT for layer A is lower than the cSTT for layer B, preferably where the difference in cSTT between layers A and B is at least 5.0, preferably 10.0, preferably at least 15.0, more preferably at least 20.0.

In one embodiment of the invention, layer A comprises fibres with a surface tension higher than 50 mN/m. For example, a hydrophilic additive can be added to the fibre composition, or physical treatment as plasma or corona might be performed. It can be advantageous to combine two layers A, B, adjacent to each other, where the layer A has a surface tension higher than 50 mN/m and the cSTT for layer A is lower than the cSTT for layer B, preferably where the difference in cSTT between layers A and B is at least 3.0, preferably at least 4.0, more preferably at least 6.0, with advantage at least 10.0.

### BRIEF DESCRIPTION OF THE DRAWINGS

FIG. 1 shows hydrophilic, hydrophobic and superhydrphobic behaviour,

FIGS. 2a to 2c show fibers exhibiting various degrees of swelling,

FIGS. 3a to 3c show various capillary effects based on philic properties of material,

FIG. 4a shows dry polyolefin fibers and FIG. 4b shows the same fibers in water.

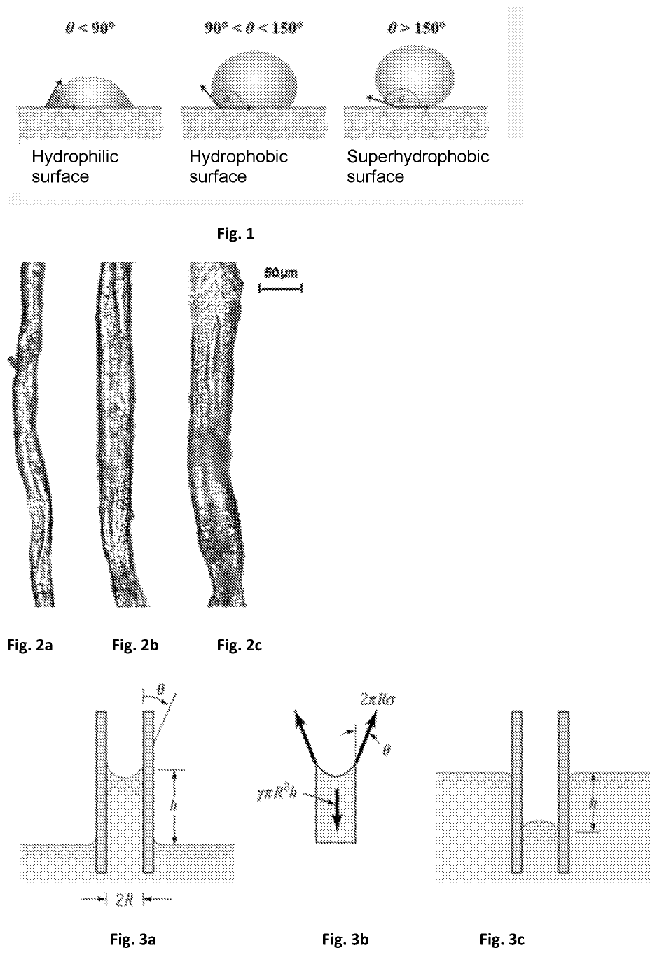

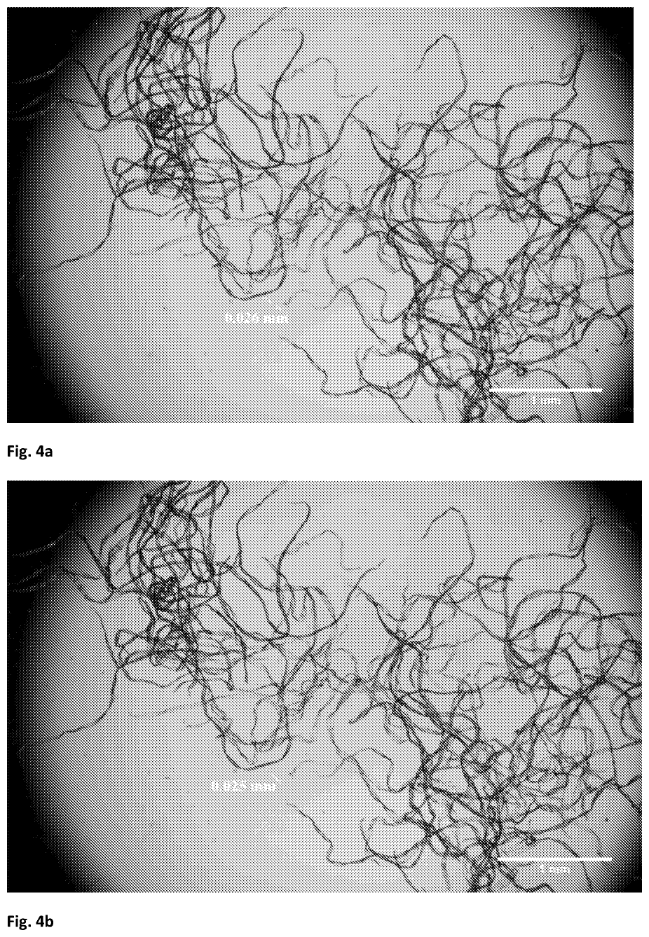

### EXAMPLE 1

A nonwoven fabric was produced using two subsequent bi-component REICOFIL spunbond beams with the same settings, with a round shape core-sheath type fibre. The core was produced from PET (Type 5520 resin from Invista) and the sheath from two different copolymers (type RT5032 from Trevira and type 701k from Invista). The process conditions and final fabric parameters for each of the Examples 1A to 1D are shown in Table 3 below.

TABLE 3

| Example | 1A | 1B | 1C | 1D |
|---|---|---|---|---|
| Basis weight set | 80 | 75 | 75 | 60 |
| [g/m²] | | | | |
| Fibre composition | PET/ | PET/ | PET/ | PET/ |
| | coPET | coPET | coPET | coPET |
| coPET type | RT5032 | 701k | 701k | RT5032 |
| | from | from | from | from |
| | Trevira | Invista | Invista | Trevira |
| Cross section | C/S | C/S | C/S | C/S |
| Mass ratio | 77:23 | 70:30 | 70:30 | 77:23 |
| Activation | 140 | 140 | 140 | 140 |
| temperature [° C.] | | | | |
| Bonding | 140 | 155 | 155 | 140 |
| temperature [° C.] | | | | |
| Bulk type | controlled | controlled | controlled | controlled |
| | shrinkage | shrinkage | shrinkage | shrinkage |
| Hydrophilic treatment | no | no | no | no |
| Thickness [mm] | 2.96 | 3.51 | 3.44 | 2.37 |
| Apparent fibre | 31.23 | 36.11 | 33.22 | 32.50 |
| diameter [μm] | | | | |
| Fibre surface energy | 45.8 | 36.7 | 36.7 | 45.8 |
| [mN/m] | | | | |
| Basis weight measured | 0.0802 | 0.0750 | 0.0756 | 0.0593 |
| [kg/m²] | | | | |
| Specific fibre | 7.49 | 6.06 | 6.64 | 5.33 |
| surface [m²/m²] | | | | |
| Void space [m³/m²] | 2.90*1e−3 | 3.46*1e−3 | 3.38*1e−3 | 2.33*1e−3 |
| cSTT | 9.68 | 9.67 | 11.97 | 4.52 |
| Measured STT | 8.52 | 9.03 | 10.77 | 3.83 |

### EXAMPLE 2

A nonwoven fabric was produced using two subsequent bi-component REICOFIL spunbond beams with the same settings, with a round shape core-sheath type fibre. The process conditions and final fabric parameters for each of the Examples 2A to 2D are shown in Table 4 below.

TABLE 4

| Example | 2A | 2B | 2C | 2D |
|---|---|---|---|---|
| Basis weight set [g/m²] | 25 | 40 | 60 | 80 |
| Fibre composition | PET/PE | PET/PE | PET/PE | PET/PE |
| Cross section | C/S | C/S | C/S | C/S |
| Mass ratio | 70:30 | 70:30 | 77:23 | 70:30 |
| Core polymer | PET | PET | PET | PET |
| | Invista | Invista | Invista | Invista |
| | 5520 | 5520 | 5520 | 5520 |
| Sheath polymer | PE Aspun | PE Aspun | PE Aspun | PE Aspun |
| | 6834 | 6834 | 6834 | 6834 |
| Activation temperature | 140 | 140 | 140 | 140 |
| [° C.] | | | | |
| Bonding temperature | 130 | 130 | 130 | 130 |
| [° C.] | | | | |
| Bulk type | controlled | controlled | controlled | controlled |
| | shrinkage | shrinkage | shrinkage | shrinkage |
| Hydrophilic treatment | no | no | no | no |
| Thickness [mm] | 0.85 | 1.39 | 1.21 | 2.33 |
| Apparent fibre diameter | 27.83 | 36.58 | 26.70 | 35.23 |
| [μm] | | | | |
| Fibre surface energy | 32.7 | 32.7 | 32.7 | 32.7 |
| [mN/m] | | | | |
| Basis weight measured | 26.0 | 40.2 | 61.5 | 78.1 |
| [kg/m²] | | | | |
| Specific fibre surface | 3.01 | 3.55 | 7.25 | 7.14 |
| [m²/m²] | | | | |
| Void space [m³/m²] | 0.83*1e−3 | 1.36*1e−3 | 1.16*1e−3 | 2.27*1e−3 |
| cSTT | 4.85 | 6.39 | 48.02 | 30.13 |
| Measured STT | 4.89 | 5.92 | over 50 | 28.70 |

### EXAMPLE 3

A nonwoven fabric was produced using two subsequent bi-component REICOFIL spunbond beams with the same settings, with a round shape side-by-side type fibre. The core was produced from PLA (type 6202 resin from Nature Works) and the sheath from PP (Tatren HT2511 from Slovnaft) polymer. The process conditions and final fabric parameters for each of the Examples 3A to 3D are shown in Table 5 below.

TABLE 5

| Example | 3A | 3B | 3C | 3D |
|---|---|---|---|---|
| Basis weight set [g/m²] | 35 | 35 | 35 | 35 |
| Fibre composition | PLA/PP | PLA/PP | PLA/PP | PLA/PP |
| Cross section | S/S | S/S | S/S | S/S |
| Mass ratio | 50:50 | 70:30 | 70:30 | 70:30 |
| Bulk type | heat | heat | heat | Heat |
| | activated | activated | activated | activated |
| | self- | self- | self- | self- |
| | crimping | crimping | crimping | crimping |
| Hydrophilic treatment | no | no | no | spin |
| | | | | finish |
| | | | | PHP10 |
| Thickness [mm] | 0.39 | 0.44 | 0.84 | 0.44 |
| Apparent fibre diameter | 13.76 | 17.45 | 31.70 | 17.45 |
| [μm] | | | | |
| Fibre surface energy | 37.85 | 41.15 | 41.15 | 55.4 |
| [mN/m] | | | | |
| Basis weight measured | 31.83 | 35.18 | 36.01 | 35.18 |
| [kg/m²] | | | | |
| Specific fibre surface | 9.22 | 6.96 | 3.92 | 6.96 |
| [m²/m²] | | | | |
| Void space [m³/m²] | 0.36*1e−3 | 0.41*1e−3 | 0.81*1e−3 | 0.40*1e−3 |
| cSTT | 91.74 | 35.57 | 5.89 | 14.68 |
| Measured STT | over 50 | 36.94 | 6.17 | 13.88 |

### EXAMPLE 4

A nonwoven fabric was produced using two subsequent bi-component REICOFIL spunbond beams with the same settings, with a round shape side-by-side type fibre. The core was produced from PLA (type 6202 resin from Nature Works) and the sheath from PE (Bio-PE SHA 7260) polymer. The process conditions and final fabric parameters for each of the Examples 4A to 4D are shown in Table 6 below.

TABLE 6

| Example | 4A | 4B | 4C | 4D |
|---|---|---|---|---|
| Basis weight set [g/m²] | 35 | 35 | 35 | 35 |
| Fibre composition | PLA/PE | PLA/PE | PLA/PE | PLA/PE |
| Cross section | eC/S | eC/S | S/S | S/S |
| Mass ratio | 70:30 | 80:20 | 70:30 | 60:40 |
| Bulk type | heat | heat | heat | heat |
| | activated | activated | activated | activated |
| | self- | self- | self- | self- |
| | crimp | crimp | crimp | crimp |
| Hydrophilic treatment | no | no | no | no |
| Thickness [mm] | 0.33 | 0.48 | 0.37 | 0.51 |
| Apparent fibre diameter | 19.37 | 40.03 | 19.37 | 32.37 |
| [μm] | | | | |
| Fibre surface energy | 34.3 | 34.3 | 41.8 | 41.8 |
| [mN/m] | | | | |
| Basis weight measured | 35.13 | 35.29 | 34.63 | 34.09 |
| [kg/m²] | | | | |
| Specific fibre surface | 6.27 | 3.13 | 6.18 | 3.74 |
| [m²/m²] | | | | |
| Void space [m³/m²] | 0.30*1e−3 | 0.45*1e−3 | 0.34*1e−3 | 0.48*1e−3 |
| cSTT | 68.57 | 11.47 | 32.32 | 8.15 |
| Measured STT | over 50 | 12.01 | 34.12 | 6.99 |

### EXAMPLE 5

The nonwoven fabric was produced using two subsequent bi-component REICOFIL spunbond beams with the same settings, with a round shape core-sheath type fibre. The core was produced from PET (Type 5520 resin from Invista). All samples were hydrophilised by means of a spin finish (PHP 90 from Schill and Seilacher) using the kiss roll. The process conditions and final fabric parameters for each of the Examples 5A to 5D are shown in Table 7 below.

TABLE 7

| Example | 5A | 5B | 5C | 5D |
|---|---|---|---|---|
| Basis weight set [g/m²] | 30 | 60 | 40 | 80 |
| Fibre composition | PET/PE | PET/PE | PET/ | PET/ |
| | | | coPET | coPET |
| Cross section | c/s | C/S | C/S | C/S |
| Mass ratio | 77:23 | 77:23 | 77:23 | 77:23 |
| Sheath polymer | PE | PE | coPET | coPET |
| | Aspun | Aspun | Trevira | Trevira |
| | 6834 | 6834 | | |
| Bulk type | con- | con- | con- | con- |
| | trolled | trolled | trolled | trolled |
| | shrink | shrink | shrink | shrink |
| Hydrophilic treatment | spin | spin | spin | spin |
| | finish | finish | finish | finish |
| | PHP 90 | PHP 90 | PHP 10 | PHP10 |
| Thickness [mm] | 0.78 | 1.34 | 1.23 | 2.86 |
| Apparent fibre diameter | 31.49 | 35.77 | 28.52 | 37.33 |
| [μm] | | | | |
| Fibre surface energy | 52.7 | 52.7 | 54.3 | 54.3 |
| [mN/m] | | | | |
| Basis weight measured | 30.1 | 60.5 | 40.6 | 83.2 |
| [kg/m²] | | | | |
| Specific fibre surface | 3.0 | 5.3 | 4.2 | 6.5 |
| [m²/m²] | | | | |
| Void space [m³/m²] | 0.75*1e−3 | 1.29*1e−3 | 1.20*1e−3 | 2.80*1e−3 |
| cSTT | 1.48 | 5.45 | 2.19 | 4.71 |
| Measured STT | 1.23 | 4.04 | 1.18 | 4.38 |

### EXAMPLE 6

Two layers were combined in one composite. The layers and their specification are shown in the table 8 below:

TABLE 8

| Example | 6A | 6B | 6C | 6D |
|---|---|---|---|---|
| Layer A | Example | Example | Example | Example |
| | 1D | 2A | 2A | 3B |
| cSTT of layer A | 4.52 | 4.85 | 4.85 | 5.89 |
| Layer B | Example | Example | Example | Example |
| | 2B | 2D | 3C | 3C |
| cSTT of layer B | 6.39 | 10.8 | 35.6 | 35.6 |
| Total basis weight | 100 | 60 | 60 | 70 |
| [kg/m²] | | | | |
| Total Specific fibre | 8.87 | 7.88 | 9.97 | 10.88 |
| surface [m²/m²] | | | | |
| Total Void space | 3.68*1e−3 | 1.45*1e−3 | 1.25*1e−3 | 1.22*1e−3 |
| [m³/m²] | | | | |
| Sum of A(cSTT) + | 11 | 16 | 40 | 41 |
| B(cSTT) | | | | |
| Measured STT | 4.9 | 8.2 | 12.8 | 14.1 |

These samples provided an excellent distribution of liquid within the layers.

In the following table two layers are combined together in one composite, layer A is a 40 gsm nonwoven fabric made from crosslinked, curled and stiffened fibres supplied by International Paper (former Weyerhaeuser). These cellulose fibres had an average thickness of 25.33 microns (fibre surface area of 4.16 m2/m2) with a surface energy of 46.4 mN/m and a thickness 2.2 mm (0.0022 m3/m2 void space), which provided the cSTT of 1.86 and also in reality the layer drew in the liquid very quickly with a dry surface after liquid absorption.

TABLE 9

| Example | 6E | 6F | 6G |
|---|---|---|---|
| Layer A | cellulose | cellulose | Example |
| | | | 2A |
| cSTT of Layer A | 1.86 | 1.86 | 1.86 |
| Layer B | Example | Example | Example |
| | 2B | 2D | 3C |
| cSTT of Layer B | 6.39 | | 35.6 |
| Total basis weight [kg/m²] | 80 | | 75 |
| Total Specific fibre | 7.71 | | 11.1 |
| surface [m²/m²] | | | |
| Total Void space [m³/m²] | 3.57*1e−3 | | 2.63*1e−3 |
| Summed A(cSTT) + B(cSTT) | 8 | | 37 |
| Measured STT | 2.7 | | 3.2 |

### EXAMPLE 7

Two layers were combined in one composite. Both layers were 60 gsm PET/PE fabrics as described in examples 5B and 2C. The layer combination is shown in the table below:

| Example | 7A | 7B | 7C |
|---|---|---|---|
| Layer A | Example 2C | Example 5B | Example 5B |
| cSTT of Layer A | 48.02 | 5.45 | 5.45 |
| Hydrophilic treatment of | no | yes | yes |
| Layer A | | | |
| Layer B | Example 2C | Example 2C | Example 5B |
| cSTT of Layer B | 48.02 | 48.02 | 5.45 |
| Hydrophilic treatment of | no | no | yes |
| Layer B | | | |
| Total basis weight [kg/m²] | 120 | 120 | 120 |
| Total Specific fibre surface | 14.8 | 12.6 | 10.6 |
| [m²/m²] | | | |
| Total Void space [m³/m²] | 2.3*1e−3 | 2.45*1e−3 | 2.58*1e−3 |
| Sum of A(cSTT) + | 96 | 53 | 11 |
| B(cSTT) | | | |
| Measured STT | over 50 | 2.67 | 1.87 |

### Testing Methodology

The “Basis weight” of a nonwoven web is measured according to the European standard test EN ISO 9073-1:1989 (conforms to WSP 130.1). There are 10 nonwoven web layers used for measurement, sample area size is 10×10 cm2.

The “Thickness” or “Calliper” of the nonwoven material is measured according to the European standard test EN ISO 9073-2:1995 (conforms to WSP 120.6) with the following modification:

1. The material shall be measured on a sample taken from production without being exposed to higher strength forces or spending more than a day under pressure (for example on a product roll), otherwise before measurement the material must lie freely on a surface for at least 24 hours.
2. The overall weight of the upper arm of the machine including added weight is 130 g. “Median fibre diameter” in a layer is expressed in SI units—micrometers (μm) or nanometers (nm). To determine the median, it is necessary to take a sample of the nonwoven fabric from at least three locations at least 5 cm away from each other. In each sample, it is necessary to measure the diameter of at least 50 individual fibres for each observed layer. It is possible to use, for example, an optical or electronic microscope (depending on the diameter of the measured fibres). In the event that the diameter of fibres in one sample varies significantly from the other two, it is necessary to discard the entire sample and to prepare a new one.

In the case of round fibres, the diameter is measured as a diameter of the cross-section of the fibres. In the event of any other shape of the fibre (e.g. hollow fibre or trilobal fibre), the cross-section surface shall be determined for each measured fibre and recalculated for a circle with same surface area. The diameter of this theoretical circle is the diameter of the fibre.

The measured values for each layer composed of all three samples are consolidated into a single set of values from which the median is subsequently determined. It applies that at least 50% of the fibres have a diameter less than or equal to the value of the median and at least 50% of the fibres have a diameter greater than or equal to the median. To identify the median of the given sample set of values, it is sufficient to arrange the values according to size and to take the value found in the middle of the list. In the event that the sample set has an even number of items, usually the median is determined as the arithmetic mean of the values in locations N/2 and N/2+1.

The “void volume” herein refers to the total amount of void space in a material relative to the bulk volume occupied by the material.

The bulk volume of the material is equal to the bulk volume of the nonwoven and can be calculated from fabric thickness (calliper) using the following equation:

bulk volume (m³)=(calliper (m))*1 (m)*1 (m)

The total amount of void space in a material can be calculated using the equation:

void space=bulk volume (m³)−mass volume (m³)

The total mass volume can be calculated using the equation:

mass volume (m³)=(weight in kilograms based on basis weight (kg))/mass density (kg/m³)

Where the mass density can be calculated from a known composition or measurement according to the norm ISO 1183-3:1999.

So the void volume can be calculated using the equation:

Void volume (%)=[1−(volume of filaments in 1 m² nonwoven fabric layer/volume of 1 m² nonwoven fabric layer)]*100%

Thus, for single component filaments:

Void volume (%)=[1−(basis weight (g/m²)/calliper (mm))/mass density (kg/m³)]*100%

Of course, when multi-component filaments are considered, wherein the components differ in density, the volume of filaments within a square meter of nonwoven fabric (NT) must be calculated accordingly.

The “recovery” of the bulkiness after the application of pressure herein refers to the ratio of the thickness of the fabric after it is freed from a load to the original thickness of the fabric. The thickness of the fabric is measured according to the EN ISO 9073-2:1995 using a preload force of 0.5 kPa). The recovery measurement procedure consists of following steps:
1. Prepare fabric samples measuring 10×10 cm
2. Measure the thickness of 1 piece of fabric
3. Measure the thickness of a pile of 5 pieces of fabric using a preload force of 0.5 kPa (Ts)
4. Load the pile of 5 fabric sheets on to a thickness meter (2.5 kPa) for 5 minutes
5. Release the weight and wait for 5 minutes
6. Measure the thickness of pile of the 5 fabrics using a preload force of 0.5 kPa (Tr)
7. Calculate the recovery according to the following equation:

Recovery=Tr/Ts(no unit)
Ts=thickness of fresh sample
Tr=thickness of recovered sample

The “compressibility” herein refers to the distance in mm by which the nonwoven is compressed by the load defined in the “resilience” measurement. It can be also be calculated as resilience (no unit)*thickness (mm). The “resilience” of a nonwoven is measured according to the European standard test EN ISO 964-1 with the following modification:
1. The thickness of one layer of the fabric is measured.
2. A pile of fabric samples is prepared so that the total thickness is at least 4 mm, optimally 5 mm in total. The pile of fabrics contains at least 1 piece of fabric.
3. The thickness of the pile of fabric samples is measured
4. A force of 5N with loading speed of 5 mm/min is applied to the pile of nonwoven samples
5. The distance of the clamp movement is measured
6. Resilience is calculated according to the equation:

R(no unit)=T1 (mm)/T0 (mm)

Or

R (%)=T1 (mm)/T0(mm)*100%

T1=distance of the clamp movement under the load 5N [mm]=how much was the pile of fabrics compressed
T0=thickness (acc. EN ISO 9073-2:1995 using the preload force of 1.06N) [mm]
The “degree of crimping” is measured according to ASTM D-3937-82 with the following modification:

1. the used unit of measurement is “crimps/cm”
Setting the degree of crimping in a bonded layer is an issue since single fibres are bonded to each other and it is not possible to remove one of them from the composition (without the danger of affecting the original crimp level) and to measure the crimp value and the fibre length. For the purpose of this invention, the following estimation may be used:
   1) A picture of the assessed layer is provided in such a magnification that the fibres can be well seen
   2) One single fibre is chosen and its path through the picture or at least part of the picture is marked
   3) The length of the marked fibre in the picture is measured
   4) The number of crimps in the measured length is counted
   In contrast to the measurement of individual fibres, it is not possible to place the fibre in such a way that all the crimps can be seen clearly and then counted in a repeating sequence. In a bonded structure, some parts may be masked in the z direction, some parts may be masked by other fibres; some parts may be masked by bonding. Each fibre turn shall be counted as half a crimp. Also, a change from sharp to blurry on one fibre shall be counted as half a crimp
   5) The number of crimps/cm is calculated

It has to be kept in mind that the value is calculated from a 2D picture of a 3D object and that the length of the fibre in the z direction is not covered. The real length of the fibre would probably be higher. Also, a 2D picture can mask certain crimps on the fibre, especially in the vicinity of a bonding point. Nevertheless, it is assumed that the described calculation can provide a relevant estimate of fibre crimping.

The “Bulk density” of a nonwoven material is calculated using the following equation:

ρ_{b}=bulk density [kg/m³]=basis weight (g/m2)/fabric thickness (mm)

BW=basis weight (acc. EN ISO 9073-1:1989) [g/m²]
T=thickness (acc. EN ISO 9073-2:1995) [mm]

The bulk density of one layer in a composite:
1) Using an optical method, the thickness of a single layer in the cross-section of a nonwoven is measured. The number of samples is at least 10 and the number is set so that the corrected sample standard deviations shall be smaller than 30% of the average value (v is below 30%)
2) The basis weight is measured
   a. The production value is taken
   b. To obtain an approximate value, it is possible to do the following:
      i. A sample of a known surface area is taken
      ii. The layers are carefully separated from each other, or the fibres from the layers are separated out,
      iii. The weight of the separated layers and the fibres from them are measured.
      iv. The basis weight is calculated from the known surface area and the weight of layer.
      v. The number of samples is at least 10 and the number is set so that the corrected sample standard deviation s is less than 20% of the average value (v is below 20%)

The corrected sample standard deviation shall be calculated using following formula:

$$s = \sqrt{\frac{1}{N - 1} {\sum }_{i = 1}^{N} {( {x}_{i} - \overline{x} )}^{2}} .$$

$$v = \frac{s}{x} \cdot 100 ( \% )$$

### Where:

N—number of samples
xi—single measured value
x—average measured value

1. A fibrous layer, wherein surface of the fibres has surface energy below 50 mN/m, characterised in that the calculated strike through time coefficient (cSTT) of the fibrous layer is below 20 and the fibrous layer is bonded in its entire volume at fibre to fibre contact bonding points, wherein
$$cSTT = \frac{{( specific fibre surface )}^{2} \times ( basis weight )}{( specific void volume ) \times {( surface energy of fibre surface )}^{3}} \times 600$$
wherein the specific fibre surface is the surface area of the fibres in m² per 1 m² of the fibrous layer,
basis weight is the weight of the layer in kg per 1 m² of the fibrous layer,
the specific void volume is the volume of empty spaces between the fibres in m³ per 1 m² of the fibrous layer.

2. The fibrous layer according to claim 1, wherein the calculated strike through time coefficient (cSTT) of the fibrous layer is below 15, preferably below 10, more preferably below 7, most preferably below 5.

3. The fibrous layer according to claim 1, wherein the basis weight of the fibrous layer is within the range of 8 to 200 gsm, and more preferably
the basis weight of the fibrous layer is more than 15 gsm, more preferably more than 20 gsm, most preferably more than 30 gsm, and/or
the basis weight of the fibrous layer is less than 150 gsm, more preferably less than 100 gsm, more preferably less than 80 gsm, most preferably less than 60 gsm.

4. The fibrous layer according to claim 1, wherein all components of the fibres of the fibrous layer are arranged across the cross-section of the fibres in a non-crimpable configuration.

5. The fibrous layer according to claim 4, wherein the fibres comprise at least one polymeric material from a group consisting of polyesters, polyamides and their blends.

6. The fibrous layer according to claim 5, wherein the fibres comprise at least one polymeric material from a group consisting of PET, coPET, PLA, coPLA and their blends.

7. The fibrous layer according to claim 1, wherein all components of the fibres of the fibrous layer are arranged across the cross section of the fibres in a crimpable configuration.

8. The fibrous layer according to claim 7, wherein the fibres comprise at least one polymeric material from a group consisting of polyesters, polyamides and their blends.

9. The fibrous layer according to claim 8, wherein the fibres comprise at least one polymeric material from a group consisting of PET, coPET, PLA, coPLA, PP, PE, PP/PE copolymer, and their blends.

10. The fibrous layer according to claim 1, wherein the fibres are crosslinked cellulose fibres.

11. A fabric comprising the fibrous layer according to claim 1, wherein the fibrous layer forms a first fibrous layer (A) and the fabric comprises a second fibrous layer (B) arranged adjacent the first fibrous layer (A), wherein the difference between the calculated strike through time coefficient cSTT of the first fibrous layer (A) and of the second fibrous layer (B) is at least 0.5, preferably at least 1.0, more preferably at least 1.5, most preferably at least 2.0.

12. The fabric according to the claim 11, wherein the first fibrous layer (A) comprises crosslinked cellulose fibres.

13. A fibrous structure comprising at least two layers, one of them comprising cellulosic crosslinked, stiffened and curled fibres and another one comprising synthetic fibres, wherein
the cellulosic fibres exhibit fibrils in their cross section and the synthetic fibres comprise homogeneous polymer or polymers in its cross section, and
the cellulosic fibres have an average length of maximum 8 mm or less and
the synthetic fibres have an average length larger than 80 mm and
at least one of the layers contains bonding material.

14. The fibrous structure according to the claim 13, wherein the synthetic fibres are endless spunbond multicomponent filaments.

15. The fibrous structure according to claim 13, wherein the bonding material is comprised in the fibres or filaments of any layer, preferably the bonding material is present as a component of surface of these fibres or filaments.

16. The fibrous structure according to claim 13, wherein the bonding material is in the form of powder binder added into one or more fibrous layers or in between them.

17. A fabric comprising at least two fibrous layers (A, B), wherein the first layer (A) comprises hydrophilic spin finish at least partially soluble in water solution on at least a part of fibre surface and the cSTT for the first layer (A) is lower than cSTT for the second layer (B), preferably wherein the difference in cSTT for the layers (A, B) is at least 5.0, preferably at least 10.0, preferably at least 15.0, more preferably at least 20.0,
wherein for each fibrous layer
$$cSTT = \frac{{( specific fibre surface )}^{2} \times ( basis weight )}{( specific void volume ) \times {( surface energy of fibre surface )}^{3}} \times 600$$
wherein the specific fibre surface is the surface area of the fibres in m² per 1 m² of the fibrous layer,
basis weight is the weight of the layer in kg per 1 m² of the fibrous layer,
the specific void volume is the volume of empty spaces between the fibres in m³ per 1 m² of the fibrous layer.

18. An absorbent article, comprising topsheet, backsheet and at least one intermediate nonwoven fibrous layer arranged between the topsheet and the backsheet and comprising polymeric superabsorbent particles, wherein at least one of the topsheet, backsheet and the intermediate nonwoven fibrous layer is formed by a fibrous layer according to claim 1.
